(19) United States
(12) Patent Application Publication
(10) Pub. No.: US 2012/0021988 A1
(43) Pub. Date: Jan. 26, 2012

(54) METHODS FOR TREATING IMMUNE THROMBOCYTOPENIA

(76) Inventors: Neil M. Bodie, Agoura Hills, CA (US); Elliot Altman, Rockvale, TN (US)

(21) Appl. No.: 12/672,615

(22) PCT Filed: Aug. 14, 2008
(86) PCT No.: PCT/US08/73173

§ 371 Date: Oct. 12, 2011

Related U.S. Application Data
(60) Provisional application No. 60/964,597, filed on Aug. 14, 2007.
(60) Provisional application No. 60/966,205, filed on Aug. 27, 2007.

(51) Int. Cl. A61K 38/10; A61P 37/06; A61P 7/00

(52) U.S. Cl. 514/13.5

(57) ABSTRACT

Polypeptides and other compounds that can bind specifically to the C_{H}2-C_{H}3 cleft of an immunoglobulin molecule, and methods for using such polypeptides and compounds to inhibit Fc-mediated immune complex formation in autoimmune/immune thrombocytopenia.

### CROSS-REFERENCE TO RELATED APPLICATIONS

This application claims priority from U.S. Provisional Application Ser. No. 60/964,597, filed Aug. 14, 2007, and U.S. Provisional Application Ser. No. 60/966,205, filed Aug. 27, 2008.

### TECHNICAL FIELD

This document relates to inhibition of immune complex formation, and more particularly to inhibition of immune complex formation associated with, for example, immune thrombocytopenia. In particular, this document relates to inhibition of immune complex formation by polypeptides and other small molecules.

### BACKGROUND

Humoral immune responses are triggered when an antigen binds specifically to an antibody. The combination of an antibody molecule and an antigen forms a small, relatively soluble immune complex (IC). Antigens either can be foreign substances, such as viral or bacterial polypeptides, or can be “self-antigens” such as polypeptides normally found in the human body. The immune system normally distinguishes foreign antigens from self-antigens. “Autoimmune” disease can occur, however, when this system breaks down, such that the immune system turns upon the body and destroys tissues or organ systems as if they were foreign substances. Larger immune complexes are more pathogenic than small, more soluble immune complexes. The formation of large, relatively insoluble immune complexes can result from both the interaction of antibody molecules with antigen and the interaction of antibody molecules with each other. Such immune complexes also can result from interactions between antibodies in the absence of antigen.

Antibodies can prevent infections by coating viruses or bacteria, but otherwise are relatively harmless by themselves. In contrast, organ specific tissue damage can occur when antibodies combine with antigens and the resulting immune complexes bind to certain effector molecules in the body. Effector molecules are so named because they carry out the pathogenic effects of immune complexes. By inhibiting the formation of large, insoluble immune complexes, or by inhibiting the binding of immune complexes to effector molecules, the tissue damaging effects of immune complexes could be prevented.

### SUMMARY

This document is based in part on the discovery that polypeptides having amino acid sequences based on those set forth in SEQ ID NO:2 (e.g., SEQ ID NO:20, also referred to herein as NB-406) can bind specifically and with high affinity to the C_{H}2-C_{H}3 domain of an immunoglobulin molecule, thus inhibiting the formation of insoluble immune complexes containing antibodies and antigens, and preventing the binding of such complexes to effector molecules. This document provides such polypeptides, as well as methods for using the polypeptides and compounds to inhibit immune complex formation and treat autoimmune complex disorders such as Idiopathic/Immune/Autoimmune Thrombocytopenia (ITP/AITP).

In one aspect, this document features a method for inhibiting immune complex formation in a subject, comprising administering to the subject a composition comprising a purified polypeptide, the polypeptide comprising the amino acid sequence (Xaa₁)ₘ-Cys-Ala-Xaa₂-His-Leu-Gly-Glu-Leu-Val-Trp-Cys-Thr-(Xaa₃)ₙ (SEQ ID NO:53), wherein Xaa₁ is any amino acid, Xaa₂ is Trp, Tyr or Phe, 5-Hydroxytrphophan (5-HTP), 5-hydroxytryptamine (5-HT), or another amino acid derivative, Xaa₃ is any amino acid, and m and n independently are 0, 1, 2, 3, 4, or 5, and wherein said immune complex formation is associated with an immune thrombocytopenia (e.g., autoimmune thrombocytopenia). The polypeptide can inhibit binding of ITP IgG Fc to an FcγR (e.g., FcγI, FcγIIa, FcγIIb/c, FcγIIIa, FcγIIIb, or FcRn). The polypeptide can inhibit binding of ITP IgG Fc to mC1q or sC1.

The polypeptide can include a terminal stabilizing group. The terminal stabilizing group can be at the amino terminus of the polypeptide and can be a tripeptide having the amino acid sequence Xaa-Pro-Pro, wherein Xaa is any amino acid (e.g., Ala). The terminal stabilizing group can be at the carboxy terminus of said polypeptide and can be a tripeptide having the amino acid sequence Pro-Pro-Xaa, wherein Xaa is any amino acid (e.g., Ala).

The method can further comprise the step of monitoring the subject for one or more clinical, histiopathological or molecular characteristics of ITP. The one or more clinical, histiopathological, or molecular characteristics of ITP can be a decrease in platelet count.

The polypeptide can include the amino acid sequence Xaa-Pro-Pro-Asp-Cys-Ala-Trp-His-Leu-Gly-Glu-Leu-Val-Trp-Cys-Thr (SEQ ID NO:19), wherein Xaa is any amino acid. For example, the polypeptide can include the amino acid sequence Ala-Pro-Pro-Asp-Cys-Ala-Trp-His-Leu-Gly-Glu-Leu-Val-Trp-Cys-Thr (SEQ ID NO:20), or the amino acid sequence Asp-Cys-Ala-Trp-His-Leu-Gly-Glu-Leu-Val-Trp-Cys-Thr (SEQ ID NO: 2).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing platelet counts in mice having induced ITP and treated with a peptide having the sequence set forth in SEQ ID NO:20 (Peptide AP) or a control peptide having the sequence set forth in SEQ ID NO:57 (Peptide CP), as indicated.

### DETAILED DESCRIPTION

This document provides polypeptides and other compounds capable of interacting with the C_{H}2-C_{H}3 cleft of an immunoglobulin molecule, such that interaction of the immunoglobulin with other molecules (e.g., effectors or other immunoglobulins) is blocked. Methods for identifying such polypeptides and other compounds also are described, along with compositions and articles of manufacture containing the polypeptides and compounds. In addition, this document provides methods for using the polypeptides and compounds to inhibit immune complex formation and to treat diseases (e.g., immune disorders such as immune/autoimmune thrombocytopenia) in which IgG immune complexes bind to effector molecules, such as membrane bound C1q (mC1q), soluble C1q (sC1q), and FcγRs (including, but not limited to FcγRI (and isoforms of FcγRs), FcγRIIa, FcγRIIb/c, FcγRIIIa, FcγRIIIb, and FcRn), which are mediators of idiopathic/immune/autoimmune thrombocytopenia (ITP/AITP).

### Immunoglobulins

The immunoglobulins make up a class of proteins found in plasma and other bodily fluids that exhibit antibody activity and bind to other molecules (e.g., antigens and certain cell surface receptors) with a high degree of specificity. Based on their structure and biological activity, immunoglobulins can be divided into five classes: IgM, IgG, IgA, IgD, and IgE. IgG is the most abundant antibody class in the body; this molecule assumes a twisted “Y” shape configuration. With the exception of the IgMs, immunoglobulins are composed mainly of four peptide chains that are linked by several intrachain and interchain disulfide bonds. For example, the IgGs are composed of two polypeptide heavy chains (H chains) and two polypeptide light chains (L chains), which are coupled by disulfide bonds and non-covalent bonds to form a protein molecule with a molecular weight of approximately 150,000 daltons (Saphire et al. (2001) Science 293:1155-1159). The average IgG molecule contains approximately 4.5 interchain disulfide bonds and approximately 12 intrachain disulfide bonds (Frangione and Milstein (1968) J. Mol. Biol. 33:893-906).

The light and heavy chains of immunoglobulin molecules are composed of constant regions and variable regions (see, e.g., Padlan (1994) Mol. Immunol. 31:169-217). For example, the light chains of an IgG1 molecule each contain a variable domain (V_{L}) and a constant domain (C_{L}). The heavy chains each have four domains: an amino terminal variable domain (V_{H}), followed by three constant domains (C_{H}1, C_{H}2, and the carboxy terminal C_{H}3). A hinge region corresponds to a flexible junction between the C_{H}1 and C_{H}2 domains. Papain digestion of an intact IgG molecule results in proteolytic cleavage at the hinge and produces an Fc fragment that contains the C_{H}2 and C_{H}3 domains, and two identical Fab fragments that each contain a C_{H}1, C_{L}, V_{H}, and V_{L} domain. The Fc fragment has complement- and tissue-binding activity, while the Fab fragments have antigen-binding activity.

Immunoglobulin molecules can interact with other polypeptides through various regions. The majority of antigen binding, for example, occurs through the V_{L}/V_{H} region of the Fab fragment. The hinge region also is thought to be important, as immunological dogma states that the binding sites for Fc receptors (FcR) are found in the hinge region of IgG molecules (see, e.g., Raghavan and Bjorkman (1996) Annu. Rev. Dev. Biol. 12:181-200). More recent evidence, however, suggests that FcR interacts with the hinge region primarily when the immunoglobulin is monomeric (i.e., not immune-complexed). Such interactions typically involve the amino acids at positions 234-237 of the Ig molecule (Wiens et al. (2000) J. Immunol. 164:5313-5318).

Immunoglobulin molecules also can interact with other polypeptides through a cleft within the C_{H}2-C_{H}3 domain. The “C_{H}2-C_{H}3 cleft” typically includes the amino acids at positions 251-255 within the C_{H}2 domain and the amino acids at positions 424-436 within the C_{H}3 domain. As used herein, numbering is with respect to an intact IgG molecule as in Kabat et al. (Sequences of Proteins of Immunological Interest, 5^{th} ed., Public Health Service, U.S. Department of Health and Human Services, Bethesda, Md.). The corresponding amino acids in other immunoglobulin classes can be readily determined by those of ordinary skill in the art.

The C_{H}2-C_{H}3 cleft is unusual in that it is characterized by both a high degree of solvent accessibility and a predominantly hydrophobic character, suggesting that burial of an exposed hydrophobic surface is an important driving force behind binding at this site. A three-dimensional change occurs at the IgG C_{H}2-C_{H}3 cleft upon antigen binding, allowing certain residues (e.g., a histidine at position 435) to become exposed and available for binding. Direct evidence of three-dimensional structural changes that occur upon antigen binding was found in a study using monoclonal antibodies sensitive to conformational changes in the Fc region of human IgG. Five IgG epitopes were altered by antigen binding: two within the hinge region and three within the C_{H}2-C_{H}3 cleft (Girkontraite et al. (1996) Cancer Biother. Radiopharm. 11:87-96). Antigen binding therefore can be important for determining whether an immunoglobulin binds to other molecules through the hinge or the Fc C_{H}2-C_{H}3 region.

The Fc region can bind to a number of effector molecules and other proteins, including the following:
(1) FcRn—The neonatal Fc receptor determines the half life of the antibody molecule in the general circulation (Leach et al., (1996) J. Immunol. 157:3317-3322; Gheti and Ward (2000) Ann. Rev. Immunol. 18:739-766). Mice genetically lacking FcRn are protected from the deleterious effects of pathogenic autoantibodies due to the shortened half-life of the autoantibodies (Liu et al. (1997) J. Exp. Med. 186:777-783). The only binding site of FcRn to the IgG Fc is the IgG Fc C_{H}2-C_{H}3 cleft and HIS 435 has been shown by 3D structure and alanine scan to be essential to FcRn to IgG Fc binding (Shields et al. (2001) J. Biol. Chem. 276:6591-6604 and Martin et al. (2001) Mol. Cell, 7:867-877). Since the peptides described herein bind with high affinity to the C_{H}2-C_{H}3 cleft and HIS 435, the peptides are direct inhibitors of (immune complexed) IgG Fc to FcRn binding. An inhibitor of FcRn binding to immune complexes or to pathogenic autoantibodies would be useful in treating diseases involving pathogenic autoantibodies and/or immune complexes.
   (2) FcR—The cellular Fc Receptor provides a link between the humoral immune response and cell-mediated effector systems (Hamano et al. (2000) J. Immunol. 164:6113-6119; Coxon et al. (2001) Immunity 14:693-704; Fossati et al. (2001) Eur. J. Clin. Invest. 31:821-831). The Fcγ Receptors are specific for IgG molecules, and include FcγRI, FcγRIIa, FcγRIIb/c, and FcγRIIIa/b (and alleles, phenotypes and genotypes thereof). These isotypes bind with differing affinities to monomeric and immune-complexed IgG.
(3) C1q—The first component of the classical complement pathway is C1, which exists in blood serum as a complex of three proteins, C1q, C1r, and C1s. The classical complement pathway is activated when C1q binds to the Fc regions of antigen-bound IgG or IgM. Although the binding of C1q to a single Fc region is weak, C1q can form tight bonds to a cluster of Fc regions. At this point C1 becomes proteolytically active.

The formation of immune complexes via interactions between immunoglobulin Fc regions and other antibodies or other factors (e.g., those described above) is referred to herein as “Fc-mediated immune complex formation” or “the Fc-mediated formation of an immune complex.” Immune complexes containing such interactions are termed “Fc-mediated immune complexes.” Fc-mediated immune complexes can include immunoglobulin molecules with or without bound antigen, and typically include C_{H}2-C_{H}3 cleft-specific ligands that have higher binding affinity for immune complexed antibodies than for monomeric antibodies.

### Purified Polypeptides

As used herein, a “polypeptide” is any chain of amino acid residues, regardless of post-translational modification (e.g., phosphorylation or glycosylation). The polypeptides provided herein typically are between 10 and 50 amino acids in length (e.g., 10, 11, 12, 13, 14, 15, 20, 25, 30, 35, 40, 45, or 50 amino acids in length). Polypeptides that are between 10 and 20 amino acids in length (e.g., 10, 11, 12, 13, 14, 15, 16, 17, 18, 19, or 20 amino acids in length) can be particularly useful.

The amino acid sequences of the polypeptides provided herein are somewhat constrained, but can have some variability. For example, the polypeptides provided herein typically include the amino acid sequence Xaa₁-Cys-Ala-Xaa₂-His-Xaa₃-Xaa₄-Xaa₅-Leu-Val-Trp-Cys-Xaa₆ (SEQ ID NO:1), wherein the residues denoted by Xaaₙ can display variability. For example, Xaa₁ can be absent or can be any amino acid (e.g., Arg or Asp). Xaa₂ can be Phe, Tyr, Trp, 5-Hydroxytryptophan (5-HTP), or Arg. Xaa₃ can be any amino acid. Xaa₄ can be Gly or Ala, while Xaa₅ can be Glu or Ala. Like Xaa₁, Xaa₆ also can be absent or can be any amino acid.

In one embodiment, a polypeptide can include the amino acid sequence Asp-Cys-Ala-Trp-His-Leu-Gly-Glu-Leu-Val-Trp-Cys-Thr (SEQ ID NO:2). Alternatively, a polypeptide can include the amino acid sequence Asp-Cys-Ala-Phe-His-Leu-Gly-Glu-Leu-Val-Trp-Cys-Thr (SEQ ID NO:3) or Asp-Cys-Ala-Arg-His-Leu-Gly-Glu-Leu-Val-Trp-Cys-Thr (SEQ ID NO:4). In another embodiment, a polypeptide can include the amino acid sequence Arg-Cys-Ala-Arg-His-Leu-Gly-Glu-Leu-Val-Trp-Cys-Thr (SEQ ID NO: 5), Arg-Cys-Ala-Trp-His-Leu-Gly-Glu-Leu-Val-Trp-Cys-Thr (SEQ ID NO: 6), or Arg-Cys-Ala-Phe-His-Leu-Gly-Glu-Leu-Val-Trp-Cys-Thr (SEQ ID NO:7).

In another embodiment, a polypeptide can include the amino acid sequence Cys-Ala-Xaa-His-Leu-Gly-Glu-Leu-Val-Trp-Cys-Thr (SEQ ID NO:8), in which Xaa can be Phe, Tyr, Trp, or Arg. For example, this document provides polypeptides that include the following amino acid sequences: Cys-Ala-Phe-His-Leu-Gly-Glu-Leu-Val-Trp-Cys-Thr (SEQ ID NO:9), Cys-Ala-Trp-His-Leu-Gly-Glu-Leu-Val-Trp-Cys-Thr (SEQ ID NO:10), and Cys-Ala-Arg-His-Leu-Gly-Glu-Leu-Val-Trp-Cys-Thr (SEQ ID NO:11).

The polypeptides provided herein can be modified for use in vivo by the addition, at the amino- or carboxy-terminal end, of a stabilizing agent to facilitate survival of the polypeptide in vivo. This can be useful in situations in which peptide termini tend to be degraded by proteases prior to cellular uptake. Such blocking agents can include, without limitation, additional related or unrelated peptide sequences that can be attached to the amino- and/or carboxy-terminal residues of the polypeptide (e.g., an acetyl group attached to the N-terminal amino acid or an amide group attached to the C-terminal amino acid). Such attachment can be achieved either chemically, during the synthesis of the polypeptide, or by recombinant DNA technology using methods familiar to those of ordinary skill in the art. Alternatively, blocking agents such as pyroglutamic acid or other molecules known in the art can be attached to the amino- and/or carboxy-terminal residues, or the amino group at the amino terminus or the carboxy group at the carboxy terminus can be replaced with a different moiety.

A proline or an Xaa-Pro-Pro (e.g., Ala-Pro-Pro) sequence at the amino terminus can be particularly useful (see, e.g., WO 00/22112). For example, a polypeptide can include the amino acid sequence Xaa₁-Pro-Pro-Cys-Ala-Xaa₂-His-Leu-Gly-Glu-Leu-Val-Trp-Cys-Thr (SEQ ID NO:12), where Xaa₁ is any amino acid (e.g., Ala), and Xaa₂ is Trp, Tyr, Phe, or Arg. For example, a polypeptide can include the amino acid sequence Xaa-Pro-Pro-Cys-Ala-Trp-His-Leu-Gly-Glu-Leu-Val-Trp-Cys-Thr (SEQ ID NO:13), Ala-Pro-Pro-Cys-Ala-Trp-His-Leu-Gly-Glu-Leu-Val-Trp-Cys-Thr (SEQ ID NO:14), Xaa-Pro-Pro-Cys-Ala-Arg-His-Leu-Gly-Glu-Leu-Val-Trp-Cys-Thr (SEQ ID NO:15), Ala-Pro-Pro-Cys-Ala-Arg-His-Leu-Gly-Glu-Leu-Val-Trp-Cys-Thr (SEQ ID NO:16), Xaa-Pro-Pro-Cys-Ala-Phe-His-Leu-Gly-Glu-Leu-Val-Trp-Cys-Thr (SEQ ID NO:17), or Ala-Pro-Pro-Cys-Ala-Phe-His-Leu-Gly-Glu-Leu-Val-Trp-Cys-Thr (SEQ ID NO:18).

Alternatively, a polypeptide can include the amino acid sequence Xaa-Pro-Pro-Asp-Cys-Ala-Trp-His-Leu-Gly-Glu-Leu-Val-Trp-Cys-Thr (SEQ ID NO:19), Ala-Pro-Pro-Asp-Cys-Ala-Trp-His-Leu-Gly-Glu-Leu-Val-Trp-Cys-Thr (SEQ ID NO:20), Xaa-Pro-Pro-Asp-Cys-Ala-Arg-His-Leu-Gly-Glu-Leu-Val-Trp-Cys-Thr (SEQ ID NO:21), Ala-Pro-Pro-Asp-Cys-Ala-Arg-His-Leu-Gly-Glu-Leu-Val-Trp-Cys-Thr (SEQ ID NO:22), Xaa-Pro-Pro-Asp-Cys-Ala-Phe-His-Leu-Gly-Glu-Leu-Val-Trp-Cys-Thr (SEQ ID NO:23), Ala-Pro-Pro-Asp-Cys-Ala-Phe-His-Leu-Gly-Glu-Leu-Val-Trp-Cys-Thr (SEQ ID NO:24), Xaa-Pro-Pro-Arg-Cys-Ala-Trp-His-Leu-Gly-Glu-Leu-Val-Trp-Cys-Thr (SEQ ID NO:25), Ala-Pro-Pro-Arg-Cys-Ala-Trp-His-Leu-Gly-Glu-Leu-Val-Trp-Cys-Thr (SEQ ID NO:26), Xaa-Pro-Pro-Arg-Cys-Ala-Arg-His-Leu-Gly-Glu-Leu-Val-Trp-Cys-Thr (SEQ ID NO:27), Ala-Pro-Pro-Arg-Cys-Ala-Arg-His-Leu-Gly-Glu-Leu-Val-Trp-Cys-Thr (SEQ ID NO:28), Xaa-Pro-Pro-Arg-Cys-Ala-Phe-His-Leu-Gly-Glu-Leu-Val-Trp-Cys-Thr (SEQ ID NO:29), or Ala-Pro-Pro-Arg-Cys-Ala-Phe-His-Leu-Gly-Glu-Leu-Val-Trp-Cys-Thr (SEQ ID NO:30).

The polypeptides provided herein can have a Pro-Pro-Xaa (e.g., Pro-Pro-Ala) sequence at their carboxy termini. For example, a polypeptide can include the amino acid sequence Cys-Ala-Trp-His-Leu-Gly-Glu-Leu-Val-Trp-Cys-Thr-Pro-Pro-Xaa (SEQ ID NO:31), Cys-Ala-Trp-His-Leu-Gly-Glu-Leu-Val-Trp-Cys-Thr-Pro-Pro-Ala (SEQ ID NO:32), Cys-Ala-Arg-His-Leu-Gly-Glu-Leu-Val-Trp-Cys-Thr-Pro-Pro-Xaa (SEQ ID NO:33), Cys-Ala-Arg-His-Leu-Gly-Glu-Leu-Val-Trp-Cys-Thr-Pro-Pro-Ala (SEQ ID NO:34), Cys-Ala-Phe-His-Leu-Gly-Glu-Leu-Val-Trp-Cys-Thr-Pro-Pro-Xaa (SEQ ID NO: 35), Cys-Ala-Phe-His-Leu-Gly-Glu-Leu-Val-Trp-Cys-Thr-Pro-Pro-Ala (SEQ ID NO: 36), Asp-Cys-Ala-Trp-His-Leu-Gly-Glu-Leu-Val-Trp-Cys-Thr-Pro-Pro-Xaa (SEQ ID NO:37), Asp-Cys-Ala-Trp-His-Leu-Gly-Glu-Leu-Val-Trp-Cys-Thr-Pro-Pro-Ala (SEQ ID NO:38), Asp-Cys-Ala-Arg-His-Leu-Gly-Glu-Leu-Val-Trp-Cys-Thr-Pro-Pro-Xaa (SEQ ID NO:39), Asp-Cys-Ala-Arg-His-Leu-Gly-Glu-Leu-Val-Trp-Cys-Thr-Pro-Pro-Ala (SEQ ID NO:40), Asp-Cys-Ala-Phe-His-Leu-Gly-Glu-Leu-Val-Trp-Cys-Thr-Pro-Pro-Xaa (SEQ ID NO:41), Asp-Cys-Ala-Phe-His-Leu-Gly-Glu-Leu-Val-Trp-Cys-Thr-Pro-Pro-Ala (SEQ ID NO:42), Arg-Cys-Ala-Trp-His-Leu-Gly-Glu-Leu-Val-Trp-Cys-Thr-Pro-Pro-Xaa (SEQ ID NO:43), Arg-Cys-Ala-Trp-His-Leu-Gly-Glu-Leu-Val-Trp-Cys-Thr-Pro-Pro-Ala (SEQ ID NO:44), Arg-Cys-Ala-Arg-His-Leu-Gly-Glu-Leu-Val-Trp-Cys-Thr-Pro-Pro-Xaa (SEQ ID NO:45), Arg-Cys-Ala-Arg-His-Leu-Gly-G lu-Leu-Val-Trp-Cys-Thr-Pro-Pro-Ala (SEQ ID NO:46), Arg-Cys-Ala-Phe-His-Leu-Gly-Glu-Leu-Val-Trp-Cys-Thr-Pro-Pro-Xaa (SEQ ID NO:47), or Arg-Cys-Ala-Phe-His-Leu-Gly-Glu-Leu-Val-Trp-Cys-Thr-Pro-Pro-Ala (SEQ ID NO:48), wherein Xaa can be any amino acid.

In some embodiments, the polypeptides provided herein can include additional amino acid sequences at the amino terminus of the sequence set forth in SEQ ID NO:1, the carboxy terminus of the sequence set forth in SEQ ID NO:1, or both. For example, a polypeptide can contain the amino acid sequence Trp-Glu-Ala-Xaa₁-Cys-Ala-Xaa₂-His-Xaa₃-Xaa₄-Xaa₅-Leu-Val-Trp-Cys-Xaa₆-Lys-Val-Glu-Glu (SEQ ID NO:49), wherein the residues denoted by Xaaₙ can display variability. As for the amino acid sequence set forth in SEQ ID NO:1, Xaa₁ can be absent or can be any amino acid (e.g., Arg or Asp); Xaa₂ can be Phe, Tyr, 5-HTP, Trp, or Arg; Xaa₃ can be any amino acid; Xaa₄ can be Gly or Ala; Xaa₅ can be Glu or Ala; and Xaa₆ can be absent or can be any amino acid. In one embodiment, a polypeptide can include the amino acid sequence Trp-Glu-Ala-Asp-Cys-Ala-Xaa-His-Leu-Gly-Glu-Leu-Val-Trp-Cys-Thr-Lys-Val-Glu-Glu (SEQ ID NO:50), where Xaa is Arg, Trp, 5-HTP, Tyr, or Phe. For example, a polypeptide can include the amino acid sequence Trp-Glu-Ala-Asp-Cys-Ala-Trp-His-Leu-Gly-Glu-Leu-Val-Trp-Cys-Thr-Lys-Val-Glu-Glu (SEQ ID NO:51).

In another embodiment, a polypeptide can consist of the amino acid sequence (Xaa₁)ₘ-Xaa₂-Cys-Ala-Xaa₃-His-Xaa₄-Xaa₅-Xaa₆-Leu-Val-Trp-Cys-(Xaa₇)ₙ (SEQ ID NO:52), wherein the residues denoted by Xaa can display variability, and m and n can be, independently, integers from 0 to 10 (e.g., 0, 1, 2, 3, 4, 5, 6, 7, 8, 9, or 10). For example, Xaa₁ can be any amino acid; Xaa₂ can be absent or can be any amino acid (e.g., Arg or Asp); Xaa₃ can be Phe, Tyr, 5-HTP, Trp, or Arg; Xaa₄ can be any amino acid; Xaa₅ can be Gly or Ala; Xaa₆ can be Glu or Ala; Xaa₇ can be any amino acid; and m and n can be from 0 to 5 (e.g., 0, 1, 2, 3, 4, or 5). Alternatively, a polypeptide can consist of the amino acid sequence (Xaa₁)ₘ-Cys-Ala-Xaa₂-His-Leu-Gly-Glu-Leu-Val-Trp-Cys-Thr-(Xaa₃)ₙ (SEQ ID NO:53), wherein Xaa₁ is any amino acid, Xaa₂ is Phe or Arg, Xaa₃ is any amino acid, and m and n are, independently, integers from 0 to 5 (e.g., 0, 1, 2, 3, 4, or 5). Examples of polypeptides within these embodiments, without limitation, include polypeptides consisting of the amino acid sequence Ala-Ala-Ala-Ala-Ala-Asp-Cys-Ala-Arg-His-Leu-Gly-Glu-Leu-Val-Trp-Cys-Ala-Ala-Ala-Ala-Ala (SEQ ID NO:54), Ala-Ala-Arg-Cys-Ala-Arg-His-Leu-Gly-Glu-Leu-Val-Trp-Cys-Thr-Ala-Ala (SEQ ID NO:55), or Ala-Ala-Ala-Asp-Cys-Ala-Phe-Trp-His-Leu-Gly-Glu-Leu-Val-Trp-Cys-Thr-Ala-Ala (SEQ ID NO:56).

The amino acid sequences of the polypeptides described herein typically contain two cysteine residues. Polypeptides containing these amino acid sequences can cyclize due to formation of a disulfide bond between the two cysteine residues. A person having ordinary skill in the art can, for example, use Ellman's Reagent to determine whether a peptide containing multiple cysteine residues is cyclized. In some embodiments, these cysteine residues can be substituted with other natural or non-natural amino acid residues that can form lactam bonds rather than disulfide bonds. For example, one cysteine residue could be replaced with aspartic acid or glutamic acid, while the other could be replaced with ornithine or lysine. Any of these combinations could yield a lactam bridge. By varying the amino acids that form a lactam bridge, a polypeptide provided herein can be generated that contains a bridge approximately equal in length to the disulfide bond that would be formed if two cysteine residues were present in the polypeptide.

The polypeptides provided herein can contain an amino acid tag. A “tag” is generally a short amino acid sequence that provides a ready means of detection or purification through interactions with an antibody against the tag or through other compounds or molecules that recognize the tag. For example, tags such as c-myc, hemagglutinin, polyhistidine, or Flag® can be used to aid purification and detection of a polypeptide. As an example, a polypeptide with a polyhistidine tag can be purified based on the affinity of histidine residues for nickel ions (e.g., on a Ni-NTA column), and can be detected in western blots by an antibody against polyhistidine (e.g., the Penta-His antibody; Qiagen, Valencia, Calif.). Tags can be inserted anywhere within the polypeptide sequence, although insertion at the amino- or carboxy-terminus is particularly useful.

The term “amino acid” refers to natural amino acids, unnatural amino acids, and amino acid analogs, all in their D and L stereoisomers if their structures so allow. Natural amino acids include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val). Unnatural amino acids include, but are not limited to 5-Hydroxytrpophan, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminoisobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodesmosine, allo-isoleucine, N-methylglycine, N-methylisoleucine, N-methylvaline, norvaline, norleucine, ornithine, and pipecolic acid.

An “analog” is a chemical compound that is structurally similar to another but differs slightly in composition (as in the replacement of one atom by an atom of a different element or in the presence of a particular functional group). An “amino acid analog” therefore is structurally similar to a naturally occurring amino acid molecule as is typically found in native polypeptides, but differs in composition such that either the C-terminal carboxy group, the N-terminal amino group, or the side-chain functional group has been chemically modified to another functional group. Amino acid analogs include natural and unnatural amino acids which are chemically blocked, reversibly or irreversibly, or modified on their N-terminal amino group or their side-chain groups, and include, for example, methionine sulfoxide, methionine sulfone, S-(carboxymethyl)-cysteine, S-(carboxymethyl)-cysteine sulfoxide and S-(carboxymethyl)-cysteine sulfone. Amino acid analogs may be naturally occurring, or can be synthetically prepared. Non-limiting examples of amino acid analogs include 5-Hydroxytrpophan (5-HTP), aspartic acid-(beta-methyl ester), an analog of aspartic acid; N-ethylglycine, an analog of glycine; and alanine carboxamide, an analog of alanine. Other examples of amino acids and amino acids analogs are listed in Gross and Meienhofer, The Peptides: Analysis, Synthesis, Biology, Academic Press, Inc., New York (1983).

The stereochemistry of a polypeptide can be described in terms of the topochemical arrangement of the side chains of the amino acid residues about the polypeptide backbone, which is defined by the peptide bonds between the amino acid residues and the α-carbon atoms of the bonded residues. In addition, polypeptide backbones have distinct termini and thus direction. The majority of naturally occurring amino acids are L-amino acids. Naturally occurring polypeptides are largely comprised of L-amino acids.

D-amino acids are the enantiomers of L-amino acids and can form peptides that are herein referred to as “inverso” polypeptides (i.e., peptides corresponding to native peptides but made up of D-amino acids rather than L-amino acids). A “retro” polypeptide is made up of L-amino acids, but has an amino acid sequence in which the amino acid residues are assembled in the opposite direction of the native peptide sequence.

“Retro-inverso” modification of naturally occurring polypeptides involves the synthetic assembly of amino acids with α-carbon stereochemistry opposite to that of the corresponding L-amino acids (i.e., D- or D-allo-amino acids), in reverse order with respect to the native polypeptide sequence. A retro-inverso analog thus has reversed termini and reversed direction of peptide bonds, while approximately maintaining the topology of the side chains as in the native peptide sequence. The term “native” refers to any sequence of L-amino acids used as a starting sequence for the preparation of partial or complete retro, inverso or retro-inverso analogs.

Partial retro-inverso polypeptide analogs are polypeptides in which only part of the sequence is reversed and replaced with enantiomeric amino acid residues. Since the retro-inverted portion of such an analog has reversed amino and carboxyl termini, the amino acid residues flanking the retro-inverted portion can be replaced by side-chain-analogous α-substituted geminal-diaminomethanes and malonates, respectively. Alternatively, a polypeptide can be a complete retro-inverso analog, in which the entire sequence is reversed and replaced with D-amino acids.

This document also provides peptidomimetic compounds that are designed on the basis of the amino acid sequences of polypeptides. Peptidomimetic compounds are synthetic, non-peptide compounds having a three-dimensional conformation (i.e., a “peptide motif,”) that is substantially the same as the three-dimensional conformation of a selected peptide, and can thus confer the same or similar function as the selected peptide. Peptidomimetic compounds can be designed to mimic any of the polypeptides provided herein.

Peptidomimetic compounds that are protease resistant are particularly useful. Furthermore, peptidomimetic compounds may have additional characteristics that enhance therapeutic utility, such as increased cell permeability and prolonged biological half-life. Such compounds typically have a backbone that is partially or completely non-peptide, but with side groups that are identical or similar to the side groups of the amino acid residues that occur in the peptide upon which the peptidomimetic compound is based. Several types of chemical bonds (e.g., ester, thioester, thioamide, retroamide, reduced carbonyl, dimethylene and ketomethylene) are known in the art to be useful substitutes for peptide bonds in the construction of peptidomimetic compounds.

The interactions between a polypeptide as described herein and an immunoglobulin molecule typically occur through the C_{H}2-C_{H}3 cleft of the immunoglobulin. Such interactions are engendered through physical proximity and are mediated by, for example, hydrophobic interactions. The “binding affinity” of a polypeptide for an immunoglobulin molecule refers to the strength of the interaction between the polypeptide and the immunoglobulin. Binding affinity typically is expressed as an equilibrium dissociation constant (K_{d}), which is calculated as K_{d}=k_{off}/kₒₙ, where k_{off}=the kinetic dissociation constant of the reaction, and kₒₙ=the kinetic association constant of the reaction. K_{d} is expressed as a concentration, with a low K_{d} value (e.g., less than 100 nM) signifying high affinity. Polypeptides that can interact with an immunoglobulin molecule typically have a binding affinity of at least 1 μM (e.g., at least 500 nM, at least 100 nM, at least 50 nM, or at least 10 nM) for the C_{H}2-C_{H}3 cleft of the immunoglobulin.

Polypeptides provided herein can bind with substantially equivalent affinity to immunoglobulin molecules that are bound by antigen and to monomeric immunoglobulins. Alternatively, the polypeptides described herein can have a higher binding affinity (e.g., at least 10-fold, at least 100-fold, or at least 1000-fold higher binding affinity) for immunoglobulin molecules that are bound by antigen than for monomeric immunoglobulins. Conformational changes that occur within the Fc region of an immunoglobulin molecule upon antigen binding to the Fab region are likely involved in a difference in affinity. The crystal structures of bound and unbound NC6.8 Fab (from a murine monoclonal antibody) showed that the tail of the Fab heavy chain was displaced by 19 angstroms in crystals of the antigen/antibody complex, as compared to its position in unbound Fab (Guddat et al. (1994) J. Mol. Biol. 236-247-274). Since the C-terminal tail of the Fab region is connected to the Fc region in an intact antibody, this shift would be expected to affect the conformation of the C_{H}2-C_{H}3 cleft. Furthermore, examination of several three-dimensional structures of intact immunoglobulins revealed a direct physical connection between the Fab heavy chain and the Fc C_{H}2-C_{H}3 cleft (Harris et al. (1997) Biochemistry 36:1581-1597; Saphire et al. (2001) Science 293:1155-1159).

Molecular modeling of the C_{H}2-C_{H}3 cleft of monomeric (i.e., unbound) and immune-complexed IgG reveal that the monomeric Fc C_{H}2-C_{H}3 cleft has a closed configuration, which can prevent binding to critical amino acid residues (e.g., His 435; see, for example, O'Brien et al. (1994) Arch. Biochem. Biophys. 310:25-31; Jefferies et al. (1984) Immunol. Lett. 7:191-194; and West et al. (2000) Biochemistry 39:9698-9708). Immune-complexed (antigen-bound) IgG, however, has a more open configuration and thus is more conducive to ligand binding. The binding affinity of RF for immune-complexed IgG, for example, is much greater than the binding affinity of RF for monomeric IgG (Corper et al. (1997) Nat. Struct. Biol. 4:374; Sohi et al. (1996) Immunol. 88:636). The same typically is true for the polypeptides provided herein.

Because the polypeptides described herein can bind to the C_{H}2-C_{H}3 cleft of immunoglobulin molecules, they can be useful for blocking the interaction of other factors (e.g., FcRn, FcR, C1q, histones, MBP, SOD1 and other immunoglobulins) to the Fc region of the immunoglobulin, and thus can inhibit Fc-mediated immune complex formation. By “inhibit” is meant that Fc-mediated immune complex formation is reduced in the presence of a polypeptide provided herein, as compared to the level of immune complex formation in the absence of the polypeptide. Such inhibiting can occur in vitro (e.g., in a test tube) or in vivo (e.g., in an individual). Any suitable method can be used to assess the level of immune complex formation. Many such methods are known in the art, and some of these are described herein.

The polypeptides described herein typically interact with the C_{H}2-C_{H}3 cleft of an immunoglobulin molecule in a monomeric fashion (i.e., interact with only one immunoglobulin molecule and thus do not link two or more immunoglobulin molecules together) with a 1:2 IgG Fc to peptide stoichiometry. Interactions with other immunoglobulin molecules through the Fc region therefore are precluded by the presence of the polypeptide. The inhibition of Fc-mediated immune complex formation can be assessed in vitro, for example, by incubating an IgG molecule with a labeled immunoglobulin molecule (e.g., a fluorescently or enzyme (ELISA) labeled Fc Receptor or C1q in the presence and absence of a polypeptide, and measuring the amount of labeled immunoglobulin that is incorporated into an immune complex. Other methods suitable for detecting immune complex formation also may be used, as discussed below.

### Preparation and Purification of Polypeptides

Polypeptides can be produced by a number of methods, many of which are well known in the art. By way of example and not limitation, a polypeptide can be obtained by extraction from a natural source (e.g., from isolated cells, tissues or bodily fluids), by expression of a recombinant nucleic acid encoding the polypeptide (as, for example, described below), or by chemical synthesis (e.g., by solid-phase synthesis or other methods well known in the art, including synthesis with an ABI peptide synthesizer; Applied Biosystems, Foster City, Calif.). Methods for synthesizing retro-inverso polypeptide analogs (Bonelli et al. (1984) Int. J. Peptide Protein Res. 24:553-556; and Verdini and Viscomi (1985) J. Chem. Soc. Perkin Trans. I:697-701), and some processes for the solid-phase synthesis of partial retro-inverso peptide analogs also have been described (see, for example, European Patent number EP0097994).

This document provides isolated nucleic acid molecules encoding the polypeptides described herein. As used herein, “nucleic acid” refers to both RNA and DNA, including cDNA, genomic DNA, and synthetic (e.g., chemically synthesized) DNA. The nucleic acid can be double-stranded or single-stranded (i.e., a sense or an antisense single strand).

The term “isolated” as used herein with reference to a nucleic acid refers to a naturally-occurring nucleic acid that is not immediately contiguous with both of the sequences with which it is immediately contiguous (one at the 5′ end and one at the 3′ end) in the naturally-occurring genome of the organism from which it is derived. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences that is normally immediately contiguous with the DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

This document also provides vectors containing the nucleic acids described herein. As used herein, a “vector” is a replicon, such as a plasmid, phage, or cosmid, into which another DNA segment may be inserted so as to bring about the replication of the inserted segment. Polypeptides can be developed using phage display, for example. Methods well known to those skilled in the art may use phage display to develop the polypeptides described herein. The vectors can be, for example, expression vectors in which the nucleotides encode the polypeptides provided herein with an initiator methionine, operably linked to expression control sequences. As used herein, “operably linked” means incorporated into a genetic construct so that expression control sequences effectively control expression of a coding sequence of interest. An “expression control sequence” is a DNA sequence that controls and regulates the transcription and translation of another DNA sequence, and an “expression vector” is a vector that includes expression control sequences, so that a relevant DNA segment incorporated into the vector is transcribed and translated. A coding sequence is “operably linked” and “under the control” of transcriptional and translational control sequences in a cell when RNA polymerase transcribes the coding sequence into mRNA, which then is translated into the protein encoded by the coding sequence.

Methods well known to those skilled in the art may be used to subclone isolated nucleic acid molecules encoding polypeptides of interest into expression vectors containing relevant coding sequences and appropriate transcriptionaUtranslational control signals. See, for example, Sambrook et al., Molecular Cloning: A Laboratory Manual (2^{nd} edition), Cold Spring Harbor Laboratory, New York (1989); and Ausubel et al., Current Protocols in Molecular Biology, Green Publishing Associates and Wiley Interscience, New York (1989). Expression vectors can be used in a variety of systems (e.g., bacteria, yeast, insect cells, and mammalian cells), as described herein. Examples of suitable expression vectors include, without limitation, plasmids and viral vectors derived from, for example, herpes viruses, retroviruses, vaccinia viruses, adenoviruses, and adeno-associated viruses. A wide variety of suitable expression vectors and systems are commercially available, including the pET series of bacterial expression vectors (Novagen, Madison, Wis.), the Adeno-X expression system (Clontech), the Baculogold baculovirus expression system (BD Biosciences Pharmingen, San Diego, Calif.), and the pCMV-Tag vectors (Stratagene, La Jolla, Calif.).

Expression vectors that encode the polypeptides described herein can be used to produce the polypeptides. Expression systems that can be used for small or large scale production of polypeptides include, without limitation, microorganisms such as bacteria (e.g., E. coli and B. subtilis) transformed with recombinant bacteriophage DNA, plasmid DNA, or cosmid DNA expression vectors containing the nucleic acid molecules provided herein; yeast (e.g., S. cerevisiae) transformed with recombinant yeast expression vectors containing the nucleic acid molecules provided herein; insect cell systems infected with recombinant virus expression vectors (e.g., baculovirus) containing the nucleic acid molecules provided herein; plant cell systems infected with recombinant virus expression vectors (e.g., tobacco mosaic virus) or transformed with recombinant plasmid expression vectors (e.g., Ti plasmid) containing the nucleic acid molecules provided herein; or mammalian cell systems (e.g., primary cells or immortalized cell lines such as COS cells, CHO cells, HeLa cells, HEK 293 cells, and 3T3 L1 cells) harboring recombinant expression constructs containing promoters derived from the genome of mammalian cells (e.g., the metallothionein promoter) or from mammalian viruses (e.g., the adenovirus late promoter and the cytomegalovirus promoter), along with the nucleic acids provided herein.

The term “purified polypeptide” as used herein refers to a polypeptide that either has no naturally occurring counterpart (e.g., a peptidomimetic), or has been chemically synthesized and is thus uncontaminated by other polypeptides, or that has been separated or purified from other cellular components by which it is naturally accompanied (e.g., other cellular proteins, polynucleotides, or cellular components). Typically, the polypeptide is considered “purified” when it is at least 70%, by dry weight, free from the proteins and naturally occurring organic molecules with which it naturally associates. A preparation of purified polypeptide therefore can be, for example, at least 80%, at least 90%, or at least 99%, by dry weight, the polypeptide. Suitable methods for purifying polypeptides can include, for example, affinity chromatography, immunoprecipitation, size exclusion chromatography, and ion exchange chromatography. The extent of purification can be measured by any appropriate method, including but not limited to: column chromatography, polyacrylamide gel electrophoresis, or high-performance liquid chromatography.

### Methods of Modeling, Designing, and Identifying Compounds

This document provides methods for designing, modeling, and identifying compounds that can bind to the C_{H}2-C_{H}3 cleft of an immunoglobulin molecule and thus serve as inhibitors of Fc-mediated immune complex formation. Such compounds also are referred to herein as “ligands.” Compounds designed, modeled, and identified by these methods typically can interact with an immunoglobulin molecule through the C_{H}2-C_{H}3 cleft, and typically have a binding affinity of at least 1 μM (e.g., at least 500 nM, at least 100 nM, at least 50 nM, or at least 10 nM) for the C_{H}2-C_{H}3 cleft of the immunoglobulin. Such compounds generally have higher binding affinity (e.g., at least 10-fold, at least 100-fold, or at least 1000-fold higher binding affinity) for immune-complexed immunoglobulin molecules than for monomeric immunoglobulin molecules.

Compounds typically interact with the C_{H}2-C_{H}3 cleft of an immunoglobulin molecule in a monomeric fashion (i.e., interact with only one immunoglobulin molecule and thus do not link two or more immunoglobulin molecules together). The interactions between a compound and an immunoglobulin molecule typically involve the amino acid residues at positions 252, 253, 435, and 436 of the immunoglobulin (number according to Kabat, supra). For example, SEQ ID NO:20 (NB-406) may have hydrophobic packing with IgG Fc Met-252, Ile-253, Ser-254, His-435 and Tyr-436 (e.g., the indole ring of Trp-14 in SEQ ID NO:20 can have a hydrophobic interaction with IgG Fc His-435). Alanine substitution of IgG Fc Asn-434, His-435 or Tyr-436 can disrupt binding (ΔΔ G≧1.5 kcal/mol). In addition, alanine substitution of SEQ ID NO:20 Val-13 or Trp-14 can result in disruption of binding (ΔΔG≧2.0 kcal/mol).

The interaction between compounds and the C_{H}2-C_{H}3 cleft renders the compounds capable of inhibiting the Fc-mediated formation of immune complexes by blocking the binding of other factors (e.g., Fc:Fc interactions, FcγRs, FcRn, histones, MBP, MOG, RF, Tau protein, α-synuclein, SOD1, TNF and C1q) to the C_{H}2-C_{H}3 cleft.

Compounds identified by the methods provided herein can be polypeptides such as, for example, those described herein. Alternatively, a compound can be any suitable type of molecule that can specifically bind to the C_{H}2-C_{H}3 cleft of an immunoglobulin molecule.

By “modeling” is meant quantitative and/or qualitative analysis of receptor-ligand structure/function based on three-dimensional structural information and receptor-ligand interaction models. This includes conventional numeric-based molecular dynamic and energy minimization models, interactive computer graphic models, modified molecular mechanics models, distance geometry and other structure-based constraint models. Modeling typically is performed using a computer and may be further optimized using known methods.

Methods of designing ligands that bind specifically (i.e., with high affinity) to the C_{H}2-C_{H}3 cleft of an immunoglobulin molecule having bound antigen typically are computer-based, and involve the use of a computer having a program capable of generating an atomic model. Computer programs that use X-ray crystallography data are particularly useful for designing ligands that can interact with an Fc C_{H}2-C_{H}3 cleft. Programs such as RasMol, for example, can be used to generate a three dimensional model of a C_{H}2-C_{H}3 cleft and/or determine the structures involved in ligand binding. Computer programs such as INSIGHT (Accelrys, Burlington, Mass.), GRASP (Anthony Nicholls, Columbia University), Dock (Molecular Design Institute, University of California at San Francisco), and Auto-Dock (Accelrys) allow for further manipulation and the ability to introduce new structures.

Methods can include, for example, providing to a computer the atomic structural coordinates for amino acid residues within the C_{H}2-C_{H}3 cleft (e.g., amino acid residues at positions 252, 253, 435, and 436 of the cleft) of an immunoglobulin molecule in an Fc-mediated immune complex, using the computer to generate an atomic model of the C_{H}2-C_{H}3 cleft, further providing the atomic structural coordinates of a candidate compound and generating an atomic model of the compound optimally positioned within the C_{H}2-C_{H}3 cleft, and identifying the candidate compound as a ligand of interest if the compound interacts with the amino acid residues at positions 252, 253, 435, and 436 of the cleft. The data provided to the computer also can include the atomic coordinates of amino acid residues at positions in addition to 252, 253, 435, and 436. By “optimally positioned” is meant positioned to optimize hydrophobic interactions between the candidate compound and the amino acid residues at positions 252, 253, 435, and 436 of the C_{H}2-C_{H}3 cleft.

Alternatively, a method for designing a ligand having specific binding affinity for the C_{H}2-C_{H}3 cleft of an immunoglobulin molecule can utilize a computer with an atomic model of the cleft stored in its memory. The atomic coordinates of a candidate compound then can be provided to the computer, and an atomic model of the candidate compound optimally positioned can be generated. As described herein, a candidate compound can be identified as a ligand having specific binding affinity for the C_{H}2-C_{H}3 cleft of an immunoglobulin molecule if, for example, the compound interacts with the amino acid residues at positions 252, 253, 435, and 436 of the cleft.

Such methods have shown that monomeric (non-antigen bound) IgG Fc binds at a site distinct from the IgG Fc C_{H}2-C_{H}3 cleft, such as the lower hinge region (Wines et al. (2000) J. Immunol. 164:5313-5318), while immune complexed (antigen bound) IgG Fc binding to FcγIIa is inhibited by an IgM rheumatoid factor (RF-AN), which has been shown by 3D structure to only bind to the IgG Fc C_{H}2-C_{H}3 interface cleft (Sohi et al. (1996) Immunology 88:636-641; and Corper et al. (1997) Nature Struct. Biol. 4(5):374-381). Soluble FcγIIa inhibits the binding of immune complexed (but not monomeric, non-immune complexed) IgG Fc to RF-AN (Wines et al. (2003) Immunol. 109:246-254), and inhibitors that bind to the IgG Fc C_{H}2-C_{H}3 cleft, such as the peptides described herein, inhibit the binding of immune complexed (antigen-bound) IgG Fc to FcγRs.

Compounds also can be interactively designed from structural information of the compounds described herein using other structure-based design/modeling techniques (see, e.g., Jackson (1997) Seminars in Oncology 24:L164-172; and Jones et al. (1996) J. Med. Chem. 39:904-917).

Compounds and polypeptides also can be identified by, for example, identifying candidate compounds by computer mo as fitting spatially and preferentially (i.e., with high affinity) into the C_{H}2-C_{H}3 cleft of an immunoglobulin molecule, and then screening those compounds in vitro or in vivo for the ability to inhibit Fc-mediated immune complex formation. Suitable methods for such in vitro and in vivo screening include those described herein.

### Compositions and Articles of Manufacture

This document provides compositions and articles of manufacture that can be used in methods for treating conditions that arise from abnormal Fc-mediated immune complex formation (e.g., over-production of Fc-mediated immune complexes). The polypeptides, compounds, and compositions provided herein can be administered to a subject (e.g., a human or another mammal) having a condition, for example, that can be alleviated by modulating Fc-mediated immune complex formation and inhibit immune complexed IgG Fc to mC1q, sC1q, FcγRs, and FcRn. Typically, one or more polypeptides or compounds can be administered to a subject suspected of having a disease or condition associated with immune complex formation. Compositions generally contain one or more polypeptides and compounds described herein. A C_{H}2-C_{H}3 binding polypeptide, for example, can be in a pharmaceutically acceptable carrier or diluent, and can be administered in amounts and for periods of time that will vary depending upon the nature of the particular disease, its severity, and the subject's overall condition. Typically, the polypeptide is administered in an inhibitory amount (i.e., in an amount that is effective for inhibiting the production of immune complexes in the cells or tissues contacted by the polypeptide). The polypeptides and methods described herein also can be used prophylactically, e.g., to minimize immunoreactivity in a subject at risk for abnormal or over-production of immune complexes (e.g., a transplant recipient).

The ability of a polypeptide to inhibit Fc-mediated immune complex formation can be assessed by, for example, measuring immune complex levels in a subject before and after treatment. A number of methods can be used to measure immune complex levels in tissues or biological samples, including those that are well known in the art. If the subject is a research animal, for example, immune complex levels in the joints can be assessed by immunostaining following euthanasia. The effectiveness of an inhibitory polypeptide also can be assessed by direct methods such as measuring the level of circulating immune complexes in serum samples. Alternatively, indirect methods can be used to evaluate the effectiveness of polypeptides in live subjects. For example, reduced immune complex formation can be inferred from clinical improvement of immune mediated diseases or in vitro or in vivo models of which have been shown to be essential in the therapeutic use in treating Atherosclerosis.

Methods for formulating and subsequently administering therapeutic compositions are well known to those skilled in the art. Dosing is generally dependent on the severity and responsiveness of the disease state to be treated, with the course of treatment lasting from several days to several months, or until a cure is effected or a diminution of the disease state is achieved. Persons of ordinary skill in the art routinely determine optimum dosages, dosing methodologies and repetition rates. Optimum dosages can vary depending on the relative potency of individual polypeptides, and can generally be estimated based on EC50 found to be effective in in vitro and in vivo animal models. Typically, dosage is from 0.01 μg to 100 g per kg of body weight, and may be given once or more daily, biweekly, weekly, monthly, or even less often. Following successful treatment, it may be desirable to have the patient undergo maintenance therapy to prevent the recurrence of the disease state.

The present document provides pharmaceutical compositions and formulations that include the polypeptides and/or compounds described herein. Polypeptides therefore can be admixed, encapsulated, conjugated or otherwise associated with other molecules, molecular structures, or mixtures of compounds such as, for example, liposomes, polyethylene glycol, receptor targeted molecules, or oral, rectal, topical or other formulations, for assisting in uptake, distribution and/or absorption.

A “pharmaceutically acceptable carrier” (also referred to herein as an “excipient”) is a pharmaceutically acceptable solvent, suspending agent, or any other pharmacologically inert vehicle for delivering one or more therapeutic compounds (e.g., C_{H}2-C_{H}3 binding polypeptides) to a subject. Pharmaceutically acceptable carriers can be liquid or solid, and can be selected with the planned manner of administration in mind so as to provide for the desired bulk, consistency, and other pertinent transport and chemical properties, when combined with one or more of therapeutic compounds and any other components of a given pharmaceutical composition. Typical pharmaceutically acceptable carriers that do not deleteriously react with amino acids include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions can be administered by a number of methods, depending upon whether local or systemic treatment is desired and upon the area to be treated. Administration can be, for example, topical (e.g., transdermal, sublingual, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, C_{H}2-C_{H}3 binding polypeptides can be administered by injection or infusion into the cerebrospinal fluid, preferably with one or more agents capable of promoting penetration of the polypeptides across the blood-brain barrier.

Formulations for topical administration of C_{H}2-C_{H}3 binding polypeptides include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Nasal sprays are particularly useful, and can be administered by, for example, a nebulizer or another nasal spray device. Administration by an inhaler also is particularly useful. Conventional pharmaceutical carriers, aqueous, powder or oily bases, thickeners and the like may be necessary or desirable.

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Compositions and formulations for parenteral, intrathecal or intraventricular administration can include sterile aqueous solutions, which also can contain buffers, diluents and other suitable additives (e.g., penetration enhancers, carrier compounds and other pharmaceutically acceptable carriers).

Pharmaceutical compositions include, without limitation, solutions, emulsions, aqueous suspensions, and liposome-containing formulations. These compositions can be generated from a variety of components that include, for example, preformed liquids, self-emulsifying solids and self-emulsifying semisolids. Emulsions are often biphasic systems comprising of two immiscible liquid phases intimately mixed and dispersed with each other; in general, emulsions are either of the water-in-oil (w/o) or oil-in-water (o/w) variety. Emulsion formulations have been widely used for oral delivery of therapeutics due to their ease of formulation and efficacy of solubilization, absorption, and bioavailability.

Liposomes are vesicles that have a membrane formed from a lipophilic material and an aqueous interior that can contain the composition to be delivered. Liposomes can be particularly useful due to their specificity and the duration of action they offer from the standpoint of drug delivery. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

Polypeptides can further encompass any pharmaceutically acceptable salts, esters, or salts of such esters, or any other compound which, upon administration to an animal including a human, is capable of providing (directly or indirectly) the biologically active metabolite or residue thereof. Accordingly, for example, this document provides pharmaceutically acceptable salts of polypeptides, prodrugs and pharmaceutically acceptable salts of such prodrugs, and other bioequivalents. The term “prodrug” indicates a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the polypeptides provided herein (i.e., salts that retain the desired biological activity of the parent polypeptide without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); and salts formed with organic acids (e.g., acetic acid, citric acid, oxalic acid, palmitic acid, or fumaric acid).

Pharmaceutical compositions containing the polypeptides provided herein also can incorporate penetration enhancers that promote the efficient delivery of polypeptides to the skin of animals. Penetration enhancers can enhance the diffusion of both lipophilic and non-lipophilic drugs across cell membranes. Penetration enhancers can be classified as belonging to one of five broad categories, i.e., surfactants (e.g., sodium lauryl sulfate, polyoxyethylene-9-lauryl ether and polyoxyethylene-20-cetyl ether); fatty acids (e.g., oleic acid, lauric acid, myristic acid, palmitic acid, and stearic acid); bile salts (e.g., cholic acid, dehydrocholic acid, and deoxycholic acid); chelating agents (e.g., disodium ethylenediaminetetraacetate, citric acid, and salicylates); and non-chelating non-surfactants (e.g., unsaturated cyclic ureas). Alternatively, inhibitory polypeptides can be delivered via iontophoresis, which involves a transdermal patch with an electrical charge to “drive” the polypeptide through the dermis.

Some embodiments provided herein include pharmaceutical compositions containing (a) one or more polypeptides and (b) one or more other agents that function by a different mechanism. For example, anti-inflammatory drugs, including but not limited to nonsteroidal anti-inflammatory drugs and corticosteroids, and antiviral drugs, including but not limited to ribivirin, vidarabine, acyclovir and ganciclovir, can be included in compositions. Other non-polypeptide agents (e.g., chemotherapeutic agents) also are within the scope of this document. Such combined compounds can be used together or sequentially.

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions provided herein, such as dyes, flavoring agents, preservatives, antioxidants, opacifiers, thickening agents and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, and aromatic substances. When added, however, such materials should not unduly interfere with the biological activities of the polypeptide components within the compositions provided herein. The formulations can be sterilized if desired.

The pharmaceutical formulations, which can be presented conveniently in unit dosage form, can be prepared according to conventional techniques well known in the pharmaceutical industry. Such techniques include the step of bringing into association the active ingredients (e.g., the C_{H}2-C_{H}3 binding polypeptides provided herein) with the desired pharmaceutical carrier(s) or excipient(s). Typically, the formulations can be prepared by uniformly and bringing the active ingredients into intimate association with liquid carriers or finely divided solid carriers or both, and then, if necessary, shaping the product. Formulations can be sterilized if desired, provided that the method of sterilization does not interfere with the effectiveness of the polypeptide contained in the formulation.

The compositions described herein can be formulated into any of many possible dosage forms such as, but not limited to, tablets, capsules, liquid syrups, soft gels, suppositories, and enemas. The compositions also can be formulated as suspensions in aqueous, non-aqueous or mixed media. Aqueous suspensions further can contain substances that increase the viscosity of the suspension including, for example, sodium carboxymethylcellulose, sorbitol, and/or dextran. Suspensions also can contain stabilizers.

C_{H}2-C_{H}3 binding polypeptides can be combined with packaging material and sold as kits for reducing Fc-mediated immune complex formation. Components and methods for producing articles of manufacture are well known. The articles of manufacture may combine one or more of the polypeptides and compounds set out in the above sections. In addition, the article of manufacture further may include, for example, buffers or other control reagents for reducing or monitoring reduced immune complex formation. Instructions describing how the polypeptides are effective for reducing Fc-mediated immune complex formation can be included in such kits.

### Idiopathic/Immune/Autoimmune Thrombocytopenia (ITP/AITP)

The term “idiopathic thrombocytopenic purpura” was used in the 1950's in reference to a clinical disorder of unknown etiology associated with thrombocytopenia. The humoral nature of idiopathic thrombocytopenic purpura was established in 1951, when it was determined that thrombocytopenic purpura could be achieved by injection plasma or IgG fractions from patients with the disorder (Harrington et al. (1951) J. Lab. Clin. Med. 38:1-10). The development of immune thrombocytopenia requires two pathologic steps: 1) formation and sustained production of IgG class antibodies to self or neo-antigens, and 2) triggering of antibody effector mechanisms that lead to accelerated platelet clearance and/or platelet activation. Fc receptors for IgG (Fcγ receptors) play a major role in immune clearance, in the accelerated clearance of Ab-coated platelets, and in the therapeutic response (Rubinstein et al. (1995) Semin. Thromb. Hemost. 21:10-22; Deckmyn et al. (1995) Sem. Thromb. Hemost. 21:46-59; and Anderson et al. (1991) Br. J. Haematol. 79:75-83). Removal of the spleen and its Fcγ receptor-laden macrophages also is of major therapeutic benefit in the treatment of immune thrombocytopenia (Blanchette et al. (1998) Semin. Hematol. 35:36-51).

A second line of evidence for the importance of Fcγ receptors in immune clearance comes from studies in mice. Mice deficient in Fcγ receptors do not become thrombocytopenic when treated with anti-platelet antibodies (Clynes et al. (1995) Immunity 3:21-26).

The complexity of the human Fcγ receptor repertoire has only recently begun to be appreciated. Human macrophages express FcγRI, FcγRIIA, and FcγRIIIA on their surface. Of the three human FcγR classes, FcγRIIA is the most widely distributed FcγR among hematopoietic cells and is the only FcγR class present on platelets and megakaryocytes (Cassel et al. (1993) Mol. Immunol. 30:451-460).

FcγRI, FcγRIIA/C, and FcγRIIIA/B are activating receptors that individually are capable of mediating phagocytosis. FcγRI and FcγRIIIA/B require an FcR subunit, or γ-chain, for expression and/or for signaling for phagocytosis in vivo. In contrast, FcγRIIA does not require the γ-chain for its expression and phagocytic activity, as it possesses IgG-binding and signal transduction capabilities in the same molecule (Indik et. al. (1991) J. Clin. Invest. 88:1766-1771). It is of note that human platelets express FcγRIIA as their sole Fc receptor (Anderson et al. (1995) Semin. Thromb. Hemost. 21:1-9).

Mouse models have been helpful in examining the pathophysiology of immune clearance. FcR γ-chain knockout (KO) mice have been generated; all activating Fcγ receptor functions are abrogated in vivo in these mice (Clynes, supra). In addition, there is no evidence of immune thrombocytopenia after injection of anti-platelet antibodies in γ-chain KO mice. Major differences exist between the Fcγ receptor repertoire in mice and humans, however, most notably the absence of FcγRIIA on mouse macrophages and the absence of any mouse platelet Fcγ receptor (Mizutani et al. (1993) Blood 82:837-844).

### Methods for Using C_{H}2-C_{H}3 Binding Polypeptides to Inhibit Fc-Mediated IC Formation

C_{H}2-C_{H}3 binding polypeptides can be used in in vitro assays of Fc-mediated IC formation. Such methods are useful to, for example, evaluate the ability of a C_{H}2-C_{H}3 cleft-binding polypeptide to block Fc-mediated IC formation. In vitro methods can involve, for example, contacting an immunoglobulin molecule (e.g., an antigen bound immunoglobulin molecule) with an effector molecule (e.g., mC1q, sC1q, FcRs and FcRn, or another antibody) in the presence and absence of a polypeptide as provided herein, and determining the level of IC formation in each sample. Levels of IC formation can be evaluated by, for example, polyacrylamide gel electrophoresis with Coomassie blue or silver staining, or by co-immunoprecipitation. Such methods are known to those of ordinary skill in the art, and can be used to test the ability of a candidate polypeptide or compound to inhibit IC formation associated with an immune disorder such as IPT, for example.

Methods provided herein also can be used to inhibit IC formation in a subject, and to treat an immune disorder in a subject by inhibiting Fc-mediated IC formation. Such methods can include, for example, administering any of the polypeptides described herein, or a composition containing any of the polypeptides described herein, to a subject having or being at risk for having or developing an immune disease (e.g., ITP). For example, a method can include administering to an individual a composition containing a polypeptide that includes the amino acid sequence Cys-Ala-Trp-His-Leu-Gly-Glu-Leu-Val-Trp-Cys-Thr (SEQ ID NO:10). Alternatively, a method can include administering to a subject a polypeptide that contains the amino acid sequence Asp-Cys-Ala-Trp-His-Leu-Gly-Glu-Leu-Val-Trp-Cys-Thr (SEQ ID NO:2), Xaa-Pro-Pro-Asp-Cys-Ala-Trp-His-Leu-Gly-Glu-Leu-Val-Trp-Cys-Thr (SEQ ID NO:19; where Xaa is any amino acid), or Ala-Pro-Pro-Asp-Cys-Ala-Trp-His-Leu-Gly-Glu-Leu-Val-Trp-Cys-Thr (SEQ ID NO:20).

The invention will be further described in the following examples, which do not limit the scope of the invention described in the claims.

### EXAMPLES

In vitro assays or measuring ligand binding to the C_{H}2-C_{H}3 cleft. In vitro assays involving enzyme-linked immunosorbent assay (ELISA) have been used to demonstrate (competitive) inhibition of immune complexed IgG Fc binding to immune mediating factors such as FcRs: (FcγRI, FcγIIa, FcγRIIb/c, FcγRIIIa/b), FcRn, mC1q, and sC1q by the polypeptides and compounds described herein. See, e.g., Examples 1 and 2 herein, as well as U.S. Patent Publication Nos. 20070225231 and 20070276125, and PCT Publication No. WO2007/030475. Standardized reagents and ELISA kits are useful to reduce costs and increase the reproducibility of the experiments.

In a standard ELISA, an antigen is immunoadsorbed onto a plastic microwell. After suitable blocking and washing, a primary antibody with specificity directed toward the antigen is added to the microwell. After another wash phase, a secondary antibody that is directed toward the primary antibody and conjugated to an enzyme marker such as horseradish peroxidase (HRP) is added to the microwell. Following another wash cycle, the appropriate enzyme substrate is added. If an antigen to primary antibody to secondary antibody/HRP conjugate is formed, the conjugated enzyme catalyzes a colorimetric chemical reaction with the substrate, which is read with a microplate reader or spectrophotometer. By standardizing the levels of the antigen and secondary antibody/HRP conjugate, a titer of the primary antibody (the variable) is established. In a standard ELISA system, the primary antibody binds to the antigen through its complementarity determining regions (CDR) located in the Fab arms. Likewise, the secondary antibody/HRP conjugate binds to the primary antibody via its CDR Fab region. Because the HRP is conjugated to the Fc region of the secondary antibody, direct Fc binding is very limited or abrogated.

For this reason, a “reverse ELISA” technique has been used to assess binding of the Fc region to ligands that bind to immune complexed IgG Fc. In a reverse ELISA, the enzyme (e.g., HRP) is not covalently conjugated to the Fc portion of the secondary antibody. Rather, a preformed immune complex of peroxidase-rabbit (or mouse) anti-peroxidase IgG (“PAP” complex) is used. In this method, HRP serves both as the antigen and the enzyme marker but does not block the Fc region. In the reverse ELISA system, an Fc C_{H}2-C_{H}3 cleft binding ligand (e.g., purified human C1q) was bound to microwell plates. In the absence of competitor, PAP complexes bound to the immobilized ligand and the reaction between HRP and its substrate produces a signal. This signal was reduced by polypeptides and compounds that inhibited PAP binding to the immobilized ligand.

### Example 1

### Inhibition of C1q Binding

PAP complexes were formed mixing 2 μl of rabbit anti-peroxidase (Sigma Chemicals, St. Louis, Mo.; Cat. No. P7899) with 50 μl of peroxidase (Sigma-Aldrich, Cat. No. P6782) in 1 ml distilled water. PAP (100 μl) were pre-incubated with 100 μl of peptide for one hour, 100 μl were pre-incubated with 100 μl of peptide or human C1q (Quidel Corp., San Diego, Calif.) for one hour. The C1q/PAP and peptide/PAP mixtures (100 μl) were incubated with C1q coated plates for 30 minutes. After washing, plates were incubated with ATBS (Quidel Corp.) for 15 minutes and read at 405 nm. Results are shown in Table 1.

TABLE 1

| | SEQ | OD |
|---|---|---|
| Peptide | ID | 405 nm |
| DCAWHLGELVWCT | 2 | 1.100 |
| APPCARHLGELVWCT | 16 | 0.567 |
| DCAFHLGELVWCT | 3 | 0.859 |
| APPDCAWHLGELVWCT | 20 | 0.389 |
| APPCAFHLGELVWCT | 18 | 0.983 |
| APPCAWHLGELVWCT | 14 | 1.148 |
| Clq (negative control) | — | 0.337 |
| Positive Control | — | 2.355 |

APPDCAWHLGELVWCT (SEQ ID NO:20) resulted in the greatest inhibition of C1q binding, almost equaling C1q itself. Peptide APPCARHLGELVWCT (SEQ ID N0:16) gave the next best result.

### Example 2

### Inhibition of FcR Binding to PAP-IC by SEQ ID NO:20

Once the reverse ELISA protocol was established using the C1q assay, the assay was redesigned using FcγIIa, FcγIIb and FcγIII in place of C1q. Highly purified FcγIIa, FcγIIb and FcγIII (R&D Systems, Minneapolis, Minn.) were immunoadsorbed onto plastic microwells. After optimizing the FcγR reverse ELISA system, simple competitive inhibition experiments using polypeptides provided herein were conducted to investigate their ability to inhibit binding of immune complexes to purified FcγR.

Falcon microtiter plates were coated with 1:10 dilutions of highly purified FcγIIa, FcγIIb and FcγIII and incubated for 24 hours. The plates were washed and then blocked with 5×BSA blocking solution (Alpha Diagnostic International, San Antonio, Tex.) for 24 hours. PAP immune complexes were formed as described in Example 1. PAP (100 μl) were pre-incubated with 100 μl of peptide for one hour. PAP/peptide mixtures were added to the FcγR coated plates and incubated for one hour. After washing, plates were incubated with ABTS substrate (Quidel Corp.) for 15 minutes and read at 405 nm. Results are shown in Table 2.

TABLE 2

| | | OD₄₀₅ | | |
|---|---|---|---|---|
| | SEQ | FcγIIa | | |
| Peptide | ID | (R131 allele) | FcγIIb | FcγIIIa |
| DCAWHLGELVWCT | 2 | 0.561 | 0.532 | 0.741 |
| APPCARHLGELVWCT | 16 | 0.956 | 0.768 | 0.709 |
| DCAFHLGELVWCT | 3 | 0.660 | 0.510 | 0.810 |
| APPDCAWHLGELVWCT | 20 | 0.509 | 0.496 | 0.670 |
| APPCAFHLGELVWCT | 18 | 0.605 | 0.380 | 0.880 |
| APPCAWHLGELVWCT | 14 | 0.658 | 0.562 | 0.530 |
| Positive Control | — | 1.599 | 1.394 | 1.588 |

Peptide APPDCAWHLGELVWCT (SEQ ID NO:20) appeared to result in the greatest overall inhibition of FcR binding to PAP, followed by peptide DCAWHLGELVWCT (SEQ ID NO:2).

Experiments with SEQ ID NO:20 were repeated. Costar microtiter plates were coated with 1:10 dilutions of highly purified FcγIIa (His 131 allele aka H161), FcγIIb and FcγIIIb and incubated for 24 hours. The plates were washed and then blocked with 10 mg/ml BSA blocking solution for 24 hours. PAP immune complexes were formed as described in Example 2. PAP (100 μl) were pre-incubated with 100 μl of peptide for one hour. PAP/peptide mixtures were added to the FcγR coated plates and incubated for one hour. After washing, plates were incubated with ABTS substrate for 15 minutes and read at 405 nm. Results are shown in Table 3.

TABLE 3

| | SEQ | | | | FCγIIIb |
|---|---|---|---|---|---|
| Peptide | ID | Fcγ1 | FCγIIa* | FCγIIb/c | (NA2) |
| APPDCAWHLGELVWCT | 20 | 0.264 | 0.209 | 0.266 | 0.266 |
| Positive control | — | 2.333 | 3.509 | 2.218 | 3.060 |
| % inhibition of | — | 89 | 94 | 88 | 91 |
| IC binding | | | | | |

*H131 allele

Thus, SEQ ID NO:20 inhibited binding of all three major classes of Fc receptor (FcγI, FcγIIa/FcγIIb, and FcγIII) to soluble PAP immune complexes.

### Example 3

### In Vivo Treatment of ITP

Mice: CD-1 mice approximately 6-8 weeks of age were obtained from Charles River (Montreal). The animals were housed 3-5 per cage in filter-topped cages. The temperature and relative humidity were maintained at 72° C. and 35-70%, respectively, and a 12 hour light/dark cycle was used. The mice were fed rodent chow and water ad libitum.

Anti-platelet antibodies to induce ITP: A rabbit anti-platelet antibody (Inter-Cell Technologies, Inc., Jupiter, Fla.; Cat. No. A31440) was used to induce ITP. Each mouse received 10 μl antibody-containing serum, diluted with 190 μl PBS for injection, such that the final injection volume was 200 μl for all groups injected with anti-platelet antibody.

On the day of use, one vial of rabbit anti-platelet antibody was removed from the freezer, placed in a 22° C. water bath, and shaken. The tube was checked every 30 seconds, and when it was completely thawed it was placed in a microcentrifuge and centrifuged at 10,000×g (11,000 rpm) for 10 minutes at 4° C. The supernatant fluid was transferred to a new tube and the pellet was discarded. The supernatant was placed on ice until used or diluted, as appropriate. Unused portions of the sera were discarded.

Therapeutic treatment of ITP: IVIg was used as a standard control treatment for the ITP induced by the rabbit anti-platelet antibody. The IVIg was Gamimmune® N, 10%. Treatment with IVIg consisted of an intraperitoneal injection of 50 mg (500 μl) IVIg, a dose that was equivalent to a dose of 2,000 mg/kg body weight.

Platelet enumeration: All mice were bled via the saphenous vein. 50 μL of blood were collected into 450 μL PBS/1% EDTA. The blood was further diluted in PBS/1% EDTA. Platelets were enumerated by flow cytometry with a Guava microcapillary cytometer (Guava EasyCyteMini, Guava Technologies, Inc., Hayward, Calif.), using forward scatter (FSC) versus side scatter (SSC) to gate platelets.

Peptides used to inhibit ITP: Peptides having the amino acid sequences set forth in SEQ ID NO:2 (DCAWHLGELVWCT), SEQ ID NO:20 (APPDCAWHLGELVWCT), and a control peptide in which Trp7, Val13 and Trp14 of SEQ ID NO:20 were replaced by alanines (APPDCAAHLGELAACT; SEQ ID NO:57) were tested for their ability to inhibit ITP. Alanine substation of Val13 and Trp14 causes a decrease in binding affinity (measured by ΔΔG, where G=Gibbs Energy) of greater than 1,000 fold.

Peptides were stored at −20° C. until the day of use. On the day of use, peptides were removed from the freezer, placed in a 22° C. water bath, shaken, and checked every 30 seconds for thawing. When thawed, the peptides were placed on ice until use. For samples that received a 10× dose of peptide, the following were sequentially added to a 1.5 ml polypropylene Eppendorf tube: 623 μl PBS (pH 7.22), 35 μL rabbit anti-plaletet IgG, and 42 μl peptide, with mixing after each addition. For samples having a 100× dose of peptide, the following were added sequentially to a 1.5 ml polypropylene Eppendorf tube: 245 μl PBS (pH 7.22), 35 μL rabbit anti-plaletet IgG, and 420 μl peptide, with mixing after each addition. The peptides were incubated for at least 30 minutes at 22° C. prior to injection into mice. (A 15 minute incubation in the laboratory, transport of the mixtures to the Vivarium, and injection took roughly an additional 30 minutes.)

The final volume of each peptide mixture was 700 μL, which was sufficient for injection of 3 mice (200 μl/mouse, plus 100 μl to cover dead volume). For the 10× dose/mouse of peptide, 12 μg of peptide (12 μl of 1 mg/ml solution) was mixed with 100 μg of the rabbit anti-platelet IgG (10 μl of 10 mg/ml solution). The volume of 700 μl was about 3.5× the volume injected per mouse (200 μl). To obtain the dose of peptide and rabbit IgG for the 700 μl final volume, dose of peptide or Rabbit IgG per mouse was multiplied by 3.5 (e.g., 12 μl peptide/mouse×3.5=42 μl, 10 μl Rabbit IgG/mouse×3.5=35 μl). PBS (623 μl, pH 7.22) was added to make up the final volume 700 μl. For the 100× dose/mouse of peptide, 120 μg of peptide (120 μl of 1 mg/ml solution) was mixed with 100 μg of the rabbit anti-platelet IgG (10 μl of 10 mg/ml solution). The volume of 700 μl was about 3.5× the volume injected per mouse (200 μl). To obtain the dose of peptide and rabbit IgG for the 700 μl final volume, dose of peptide or rabbit IgG per mouse was multiplied by 3.5 (e.g., 120 μl peptide/mouse×3.5=420 μl, 10 μl rabbit IgG/mouse×3.5=35 μl). PBS (245 p. 1, pH 7.22) was added to make up the final volume 700 μl.

The experimental timing was as follows:

Day 0: Appropriate mice were injected with 50 mg IVIg

Day 1: Appropriate mice were injected with anti-platelet Ab±peptides

Day 2: All mice were bled at 24 hours for platelet counting by flow cytometry.

Each treatment group included 3 mice. Groups were as follows:
1. Control (untreated)
2. Rabbit anti-platelet IgG (i.e., ITP)
3. IVIg (day 0)+rabbit anti-platelet IgG
4. 10× molar amount SEQ ID NO:20 (Peptide AP; the ratio of the molar amount of peptide to molar amount of rabbit anti-mouse anti-platelet antibody)+rabbit anti-platelet IgG
5. 100× molar amount SEQ ID NO:20 (Peptide AP)+rabbit anti-platelet IgG
6. 10× molar amount SEQ ID NO:2 (Peptide BP)+rabbit anti-platelet IgG
7. 100× molar amount SEQ ID NO:2 (Peptide BP)+rabbit anti-platelet IgG
8. 10× Molar amount of control peptide (Peptide CP)+rabbit anti-platelet IgG
9. 100× Molar amount of control peptide (Peptide CP)+rabbit anti-platelet IgG

Peptide mixtures were injected intraperitoneally using a 30 gauge needle and a 1 ml tuberculin slip tip syringe. After 24 hours, mice were bled for platelet counting.

Results are shown in Table 4, which provides raw platelet counts (×10⁹/L). Data represent platelet counts from individual mice. Statistics were performed using the Prism 4.0 graphing package (GraphPad Software, Inc., La Jolla, Calif.). These data indicate that SEQ ID NO:20 (Peptide AP, also referred to herein as NB406), ameliorated induced ITP (with 100× molar Peptide AP having a greater effect than 10× Peptide AP), while SEQ ID NO:2 (Peptide BP) and the control (SEQ ID NO:57; Peptide CP) had no effect.

TABLE 4

| | Platelet counts (×10⁹/L | | | | | | | | |
|---|---|---|---|---|---|---|---|---|---|
| | | | | ITP | ITP | ITP | ITP | ITP | ITP |
| | Nil | | ITP | 10X | 100X | 10X | 100X | 10X | 100X |
| Animal | (control) | ITP | IVIg | AP | AP | BP | BP | CP | CP |
| 1 | 1140 | 167 | 820 | 203 | 759 | 163 | 155 | 199 | 155 |
| 2 | 867 | 182 | 893 | 261 | 642 | 117 | 125 | 191 | 160 |
| 3 | 1040 | 163 | 991 | 197 | 433 | 233 | 194 | 143 | 179 |
| mean | 1016 | 170.7 | 901.3 | 220.3 | 611.3 | 171.0 | 158.0 | 177.7 | 164.7 |
| SD | 138.1 | 10.02 | 85.80 | 35.35 | 165.1 | 58.41 | 34.60 | 30.29 | 12.66 |
| SE | 79.74 | 5.783 | 49.54 | 20.41 | 95.35 | 33.72 | 19.97 | 17.49 | 7.311 |

A second experiment was conducted in which the experimental protocol was essentially identical to the previous experiment, except that mice were treated either with SEQ ID NO:20 (Peptide AP) or SEQ ID NO:57 (the control Peptide CP). The following groups of mice (3 mice/group) were used:

1. Control (untreated)

2. 10 μl rabbit anti-platelet IgG (i.e., ITP)

3. IVIg (day 0)+10 μl rabbit anti-platelet IgG

4. 10× Peptide AP+10 μl rabbit anti-platelet IgG

5. 100× Peptide AP+10 μl rabbit anti-platelet IgG

6. 10× Peptide CP+10 μl rabbit anti-platelet IgG

7. 100× Peptide CP+10 p. 1 rabbit anti-platelet IgG

8. 3 μl rabbit anti-platelet IgG (i.e., lower level of ITP)

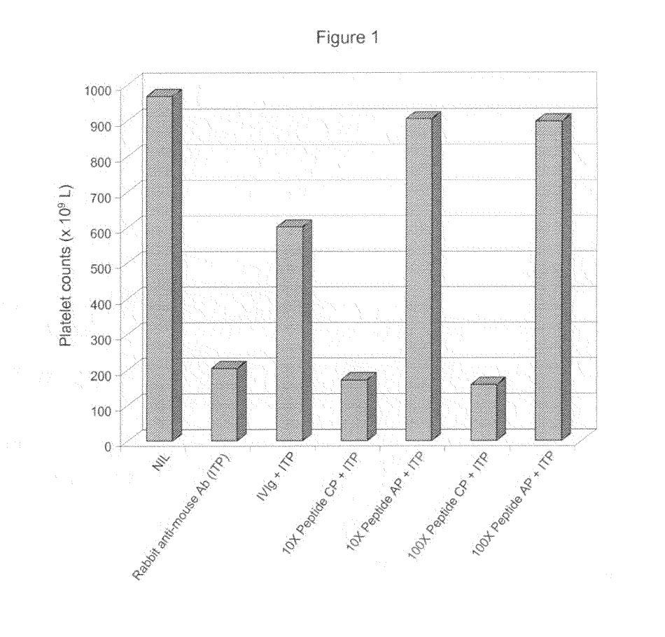

Twenty-four hours after treatment, blood samples were obtained and platelets were enumerated. Results from this experiment are shown graphically in FIG. 1. As shown, 10× molar and 100× molar Peptide AP ameliorated the induced ITP/AITP better than the currently accepted treatment for ITP (IVIg).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for inhibiting immune complex formation in a subject, said method comprising administering to said subject a composition comprising a purified polypeptide, said polypeptide comprising the amino acid sequence (Xaa₁)ₘ-Cys-Ala-Xaa₂-His-Leu-Gly-Glu-Leu-Val-Trp-Cys-Thr-(Xaa₃)ₙ (SEQ ID NO:53), wherein Xaa₁ is any amino acid, Xaa₂ is Trp, Tyr or Phe, 5-Hydroxytrphophan (5-HTP), 5-hydroxytryptamine (5-HT), or another amino acid derivative, Xaa₃ is any amino acid, and m and n independently are 0, 1, 2, 3, 4, or 5, and wherein said immune complex formation is associated with an immune thrombocytopenia (ITP).

2. The method of claim 1, wherein said immune complex formation is associated with autoimmune thrombocytopenia (AITP).

3. The method of claim 1, wherein said polypeptide inhibits binding of ITP IgG Fc to an FcγR.

4. The method of claim 3, wherein said FcγR is FcγI, FcγIIa, FcγIIb/c, FcγIIIa, FcγIIIb, or FcRn.

5. The method of claim 1, wherein said polypeptide inhibits binding of ITP IgG Fc to mC1q or sC1q.

6. The method of claim 1, wherein said polypeptide comprises a terminal stabilizing group.

7. The method of claim 6, wherein said terminal stabilizing group is at the amino terminus of said polypeptide and is a tripeptide having the amino acid sequence Xaa-Pro-Pro, wherein Xaa is any amino acid.

8. The method of claim 7, wherein Xaa is Ala.

9. The method of claim 6, wherein said terminal stabilizing group is at the carboxy terminus of said polypeptide and is a tripeptide having the amino acid sequence Pro-Pro-Xaa, wherein Xaa is any amino acid.

10. The method of claim 9, wherein Xaa is Ala.

11. The method of claim 1, further comprising the step of monitoring said subject for one or more clinical, histiopathological or molecular characteristics of ITP.

12. The method of claim 11, wherein said one or more clinical, histiopathological, or molecular characteristics of ITP is a decrease in platelet count.

13. The method of claim 1, wherein said polypeptide comprises the amino acid sequence Xaa-Pro-Pro-Asp-Cys-Ala-Trp-His-Leu-Gly-Glu-Leu-Val-Trp-Cys-Thr (SEQ ID NO:19), wherein Xaa is any amino acid.

14. The method of claim 1, wherein said polypeptide comprises the amino acid sequence Ala-Pro-Pro-Asp-Cys-Ala-Trp-His-Leu-Gly-Glu-Leu-Val-Trp-Cys-Thr (SEQ ID NO:20).

15. The method of claim 1, wherein said polypeptide comprises the amino acid sequence Asp-Cys-Ala-Trp-His-Leu-Gly-Glu-Leu-Val-Trp-Cys-Thr (SEQ ID NO: 2).


SEQUENCE LISTING (58 sequences)

SEQ ID NO: 1 - protein, 13 aa, Artificial Sequence
  Synthetic polypeptide
XCAXHXXXLVWCX

SEQ ID NO: 2 - protein, 13 aa, Artificial Sequence
  Synthetic polypeptide
DCAWHLGELVWCT

SEQ ID NO: 3 - protein, 13 aa, Artificial Sequence
  Synthetic polypeptide
DCAFHLGELVWCT

SEQ ID NO: 4 - protein, 13 aa, Artificial Sequence
  Synthetic polypeptide
DCARHLGELVWCT

SEQ ID NO: 5 - protein, 13 aa, Artificial Sequence
  Synthetic polypeptide
RCARHLGELVWCT

SEQ ID NO: 6 - protein, 13 aa, Artificial Sequence
  Synthetic polypeptide
RCAWHLGELVWCT

SEQ ID NO: 7 - protein, 13 aa, Artificial Sequence
  Synthetic polypeptide
RCAFHLGELVWCT

SEQ ID NO: 8 - protein, 12 aa, Artificial Sequence
  Synthetic polypeptide
CAXHLGELVWCT

SEQ ID NO: 9 - protein, 12 aa, Artificial Sequence
  Synthetic polypeptide
CAFHLGELVWCT

SEQ ID NO: 10 - protein, 12 aa, Artificial Sequence
  Synthetic polypeptide
CAWHLGELVWCT

SEQ ID NO: 11 - protein, 12 aa, Artificial Sequence
  Synthetic polypeptide
CARHLGELVWCT

SEQ ID NO: 12 - protein, 15 aa, Artificial Sequence
  Synthetic polypeptide
XPPCAXHLGELVWCT

SEQ ID NO: 13 - protein, 15 aa, Artificial Sequence
  Synthetic polypeptide
XPPCAWHLGELVWCT

SEQ ID NO: 14 - protein, 15 aa, Artificial Sequence
  Synthetic polypeptide
APPCAWHLGELVWCT

SEQ ID NO: 15 - protein, 15 aa, Artificial Sequence
  Synthetic polypeptide
XPPCARHLGELVWCT

SEQ ID NO: 16 - protein, 15 aa, Artificial Sequence
  Synthetic polypeptide
APPCARHLGELVWCT

SEQ ID NO: 17 - protein, 15 aa, Artificial Sequence
  Synthetic polypeptide
XPPCAFHLGELVWCT

SEQ ID NO: 18 - protein, 15 aa, Artificial Sequence
  Synthetic polypeptide
APPCAFHLGELVWCT

SEQ ID NO: 19 - protein, 16 aa, Artificial Sequence
  Synthetic polypeptide
XPPDCAWHLGELVWCT

SEQ ID NO: 20 - protein, 16 aa, Artificial Sequence
  Synthetic polypeptide
APPDCAWHLGELVWCT

SEQ ID NO: 21 - protein, 16 aa, Artificial Sequence
  Synthetic polypeptide
XPPDCARHLGELVWCT

SEQ ID NO: 22 - protein, 16 aa, Artificial Sequence
  Synthetic polypeptide
APPDCARHLGELVWCT

SEQ ID NO: 23 - protein, 16 aa, Artificial Sequence
  Synthetic polypeptide
XPPDCAFHLGELVWCT

SEQ ID NO: 24 - protein, 16 aa, Artificial Sequence
  Synthetic polypeptide
APPDCAFHLGELVWCT

SEQ ID NO: 25 - protein, 16 aa, Artificial Sequence
  Synthetic polypeptide
XPPRCAWHLGELVWCT

SEQ ID NO: 26 - protein, 16 aa, Artificial Sequence
  Synthetic polypeptide
APPRCAWHLGELVWCT

SEQ ID NO: 27 - protein, 16 aa, Artificial Sequence
  Synthetic polypeptide
XPPRCARHLGELVWCT

SEQ ID NO: 28 - protein, 16 aa, Artificial Sequence
  Synthetic polypeptide
APPRCARHLGELVWCT

SEQ ID NO: 29 - protein, 16 aa, Artificial Sequence
  Synthetic polypeptide
XPPRCAFHLGELVWCT

SEQ ID NO: 30 - protein, 16 aa, Artificial Sequence
  Synthetic polypeptide
APPRCAFHLGELVWCT

SEQ ID NO: 31 - protein, 15 aa, Artificial Sequence
  Synthetic polypeptide
CAWHLGELVWCTPPX

SEQ ID NO: 32 - protein, 15 aa, Artificial Sequence
  Synthetic polypeptide
CAWHLGELVWCTPPA

SEQ ID NO: 33 - protein, 15 aa, Artificial Sequence
  Synthetic polypeptide
CARHLGELVWCTPPX

SEQ ID NO: 34 - protein, 15 aa, Artificial Sequence
  Synthetic polypeptide
CARHLGELVWCTPPA

SEQ ID NO: 35 - protein, 15 aa, Artificial Sequence
  Synthetic polypeptide
CAFHLGELVWCTPPX

SEQ ID NO: 36 - protein, 15 aa, Artificial Sequence
  Synthetic polypeptide
CAFHLGELVWCTPPA

SEQ ID NO: 37 - protein, 16 aa, Artificial Sequence
  Synthetic polypeptide
DCAWHLGELVWCTPPX

SEQ ID NO: 38 - protein, 16 aa, Artificial Sequence
  Synthetic polypeptide
DCAWHLGELVWCTPPA

SEQ ID NO: 39 - protein, 16 aa, Artificial Sequence
  Synthetic polypeptide
DCARHLGELVWCTPPX

SEQ ID NO: 40 - protein, 16 aa, Artificial Sequence
  Synthetic polypeptide
DCARHLGELVWCTPPA

SEQ ID NO: 41 - protein, 16 aa, Artificial Sequence
  Synthetic polypeptide
DCAFHLGELVWCTPPX

SEQ ID NO: 42 - protein, 16 aa, Artificial Sequence
  Synthetic polypeptide
DCAFHLGELVWCTPPA

SEQ ID NO: 43 - protein, 16 aa, Artificial Sequence
  Synthetic polypeptide
RCAWHLGELVWCTPPX

SEQ ID NO: 44 - protein, 16 aa, Artificial Sequence
  Synthetic polypeptide
RCAWHLGELVWCTPPA

SEQ ID NO: 45 - protein, 16 aa, Artificial Sequence
  Synthetic polypeptide
RCARHLGELVWCTPPX

SEQ ID NO: 46 - protein, 16 aa, Artificial Sequence
  Synthetic polypeptide
RCARHLGELVWCTPPA

SEQ ID NO: 47 - protein, 16 aa, Artificial Sequence
  Synthetic polypeptide
RCAFHLGELVWCTPPX

SEQ ID NO: 48 - protein, 16 aa, Artificial Sequence
  Synthetic polypeptide
RCAFHLGELVWCTPPA

SEQ ID NO: 49 - protein, 20 aa, Artificial Sequence
  Synthetic polypeptide
WEAXCAXHXXXLVWCXKVEE

SEQ ID NO: 50 - protein, 20 aa, Artificial Sequence
  Synthetic polypeptide
WEADCAXHLGELVWCTKVEE

SEQ ID NO: 51 - protein, 20 aa, Artificial Sequence
  Synthetic polypeptide
WEADCAWHLGELVWCTKVEE

SEQ ID NO: 52 - protein, 32 aa, Artificial Sequence
  Synthetic polypeptide
XXXXXXXXXXXCAXHXXXLVWCXXXXXXXXXX

SEQ ID NO: 53 - protein, 22 aa, Artificial Sequence
  Synthetic polypeptide
XXXXXCAXHLGELVWCTXXXXX

SEQ ID NO: 54 - protein, 22 aa, Artificial Sequence
  Synthetic polypeptide
AAAAADCARHLGELVWCAAAAA

SEQ ID NO: 55 - protein, 17 aa, Artificial Sequence
  Synthetic polypeptide
AARCARHLGELVWCTAA

SEQ ID NO: 56 - protein, 19 aa, Artificial Sequence
  Synthetic polypeptide
AAADCAFWHLGELVWCTAA

SEQ ID NO: 57 - protein, 16 aa, Artificial Sequence
  Synthetic polypeptide
APPDCAAHLGELAACT

SEQ ID NO: 58 - protein, 22 aa, Artificial Sequence
  Synthetic polypeptide
XXXXXCAXHLGELVWCTXXXXX